(19) United States
(12) Patent Application Publication
(10) Pub. No.: US 2026/0354990 A1
(43) Pub. Date: Nov. 12, 2026

(54) Orthopedic joint device

(71) Applicant: OTTO BOCK HEALTHCARE PRODUCTS GMBH, Wien (AT)

(72) Inventor: Reinold Philipp DURSTBERGER, Wien (AT)

(21) Appl. No.: 19/102,026

(22) PCT Filed: Aug. 2, 2023
(86) PCT No.: PCT/EP2023/071445

§ 371 Date: Feb. 7, 2025

(30) Foreign Application Priority Data
Aug. 8, 2022 (DE) 10 2022 119 920.7

(51) Int. Cl. A61F 2/58; A61F 2/50; A61F 2/66

(52) U.S. Cl. CPC A61F 2/585; A61F 2/6607; A61F 2002/503; A61F 2002/5096

(57) ABSTRACT

An orthopedic joint device having an upper part and a lower part between which a joint is formed, wherein: the joint defines at least two swivel axes that are not parallel to each other and at least one restoring element is fastened to the upper part and the lower part; and the restoring element has an elastic element which exerts a restoring force in the direction of an initial position when the upper part is swiveled relative to the lower part from the initial position.

The invention relates to an orthopedic joint device having an upper part and a lower part, between which a joint is formed.

An orthopedic joint device is in particular an articulated connection between an upper part and a lower part in prostheses, orthoses and exoskeletons. In addition to providing the possibility of moving the upper part and the lower part relative to each other and of enabling a pivoting movement of the upper part relative to the lower part, the joint device also serves to hold the upper part on the lower part, such that a geometric assignment of upper part and lower part to each other is maintained.

Orthopedic joint devices can be constructed in different ways. A comparatively simple design is one in which the upper part is fastened pivotably to the lower part via a rigid shaft. Such an embodiment is found, for example, in single-axis prosthetic knee joints or orthoses. In what are called multi-link joints, for example in prosthetic knee joints with four or more joint components which are articulated with respect to one another via four or correspondingly more axes, a pivoting movement of the upper part relative to the lower part is likewise permitted about a single pivot axis; however, this pivot axis is not stationary and migrates on a path during the pivoting movement. The upper part has only one rotational degree of freedom relative to the lower part and moves, for example, on a circular path, so that two translational degrees of freedom are present. In a simple single-axis joint, there is only one rotational degree of freedom, but no translational degree of freedom.

EP 1 962 734 B1 discloses a prosthetic knee joint having a chassis which is fastened to a mounting plate. The mounting plate is mounted on a coupling element pivotably about a pivot axis. The coupling element for its part is fastened to a housing. Arranged between the mounting plate and the coupling element is a spring element, which provides a restoring force upon each deflection about the axis of rotation. The housing can also be mounted rotatably about a second axis, which runs perpendicular to the longitudinal extent of a forearm. The prosthetic hand thus has two joint devices, one between the mounting plate and the coupling element, and one between the housing and the forearm shaft. The rotations about the two pivot axes can be blocked by separate blocking devices. In an unblocked state, flexion and extension are possible against a counterforce, and rotation about the longitudinal axis of the forearm shaft is possible without counterforce.

The object of the present invention is to provide an orthopedic joint device with which an improved natural appearance can be achieved.

This object is achieved by an orthopedic joint device having the features of the independent claim. Advantageous embodiments and developments of the invention are disclosed in the dependent claims, the description and the figures.

In the orthopedic joint device having an upper part and a lower part between which a joint is formed, provision is made that the joint forms at least two pivot axes, which are not oriented parallel to each other, and that at least one restoring element is fastened to the upper part and the lower part, wherein the restoring element has an elastic element which, during a pivoting movement of the upper part relative to the lower part from a starting position, exerts a restoring force in the direction of the starting position. The joint is formed between the two components directly assigned to each other, namely the upper part and the lower part, and by said two components assigned to each other, and has two pivot axes about which the upper part can be pivoted relative to the lower part. These two pivot axes are not oriented parallel to each other and, in one embodiment, can be designed in such a way that the two pivot axes intersect. The joint device has at least one restoring element, which is preferably elastic or at least has an elastic component or an elastic element, wherein the restoring element is fastened both to the upper part and to the lower part. By the fastening to an upper part and to the lower part, it is possible to transmit forces between the upper part and the lower part via the restoring element. When the upper part pivots relative to the lower part from a starting position, in which the upper part is advantageously held to the lower part by the restoring element, a restoring force in the direction of the starting position is exerted upon elongation of the restoring element by the elastic element, such that the starting position is adopted again when there is no force acting from outside. With a design of this kind, it is possible that the upper part can pivot relative to the lower part about two pivot axes without having to move further components, and it is moved back to the starting position so that, for example in the case of a wrist, not only can a palmar flexion and dorsal extension take place, but also a radial abduction and an ulnar abduction. For example, movements about a dorsopalmar axis and about a radioulnar axis are possible. In an ankle joint, simultaneous plantar flexion or dorsal extension would be possible together with supination or pronation. In principle, it is also possible to provide more than two pivot axes for the joint.

In one embodiment, the joint has a joint head and a joint socket which, for example, bear directly on each other. Between the joint socket and the joint head, an intermediate element can also be arranged in the joint gap, for example in order to reduce friction, to dampen shocks and/or to compensate for surface irregularities. The joint can be formed as a ball joint or ellipsoid joint. With a ball joint, three pivot axes or three rotational degrees of freedom can be achieved, which permit a free rotational mobility of the upper part relative to the lower part. The rotational restrictions are then effected via the restoring forces applied by the restoring element or the restoring elements, wherein different levels of restoring forces can be provided in order to achieve different stabilities and resistances against the respective pivoting movement about the respective axis. In one embodiment, the joint is formed as an ellipsoid joint or what is called a condyloid joint, in which a rotational degree of freedom is blocked, or, on account of the design of the joint socket and the joint head and of the holding force of the restoring element, by which the joint head is pressed into the joint socket, a pivoting movement about a third rotational degree of freedom is made more difficult. In the case of a barrel-like embodiment of the joint head and of the joint socket in a prosthetic wrist, a dorsal extension and palmar flexion would be possible, just as a radial abduction and ulnar abduction, a pronation or supination of the hand relative to the forearm would not be possible. However, in the case of a very flat embodiment of the joint socket, or when high forces are applied, supination or pronation of a prosthetic wrist with a corresponding structural design of the restoring elements would be possible in principle. If necessary, the joint head will then be lifted from the joint socket, or an otherwise planar or linear loading of joint head and joint socket would be canceled. However, supination or pronation in the joint is effected under difficult structural conditions.

In one embodiment, the pivot axes of the joint are oriented perpendicular to each other, so that a simple combined movement about the two pivot axes can also take place. The pivot axes do not have to intersect each other; in the ellipsoid joint, the two radii about the axes can be of different sizes. In a ball joint, all three possible pivot axes are perpendicular to one another and intersect at the center of the ball, which is defined by the ball cap or the ball segment of the joint head or of the joint socket.

In one embodiment, the joint has a translational degree of freedom, in particular exactly one translational degree of freedom, so that the joint gap between the joint head and the joint socket can increase, such that a displacement in the two other translational degrees of freedom is however prevented or at least made difficult. The joint socket can then be lifted off from the joint head if no holding forces are applied, for example by the restoring elements, while a displacement in the other two directions is not possible. This gives the joint greater stability. In principle, there is also the possibility that the joint socket is mounted so as also to be movable in translation relative to the joint head in another or several other planes or that, with correspondingly low prestressing of the restoring element or of the elastic portion of the restoring element, it permits a combined translational movement in several planes or directions.

In one embodiment, a plurality of restoring elements with an elastic element or a plurality of elastic elements, which are formed or arranged thereon, are fastened to the upper part and the lower part and are arranged to act in different directions, in particular in opposite directions. In the case of a wrist, for example, restoring elements are arranged on the palmar and dorsal aspects in order to apply a restoring force to a dorsal extension and a palmar flexion. To permit radial abduction and ulnar abduction and at the same time to enable restoring to a starting position, restoring elements are arranged medially and laterally between the upper part and the lower part, for example. A corresponding arrangement occurs in an ankle joint with restoring elements against plantar flexion and dorsal flexion and also supination and pronation. It is also possible to provide only one restoring element for each of the different pivot axes, which restoring element(s) can be loaded in both the tension direction and the pressure direction and provide corresponding restoring forces, for example by tension-compression springs, elastomer elements or the like.

In one embodiment, the restoring element or the restoring elements is/are formed as a tension element, belt, band or elastomer or has/have at least one elastic component. The elastic component can be, for example, an elastic belt portion, an elastic band portion, or a spring that is arranged on or in the restoring element. In addition, the restoring element has at least one tensionally rigid, flexible component, which is preferably arranged or formed in the distal region of the restoring element. The tensionally rigid, flexible component can also extend over the entire length of the restoring element and can be arranged or activated parallel thereto. If the tensionally rigid component is fixed relative to the upper part and the lower part, a displacement of the lower part relative to the upper part that would cause deflection of the tensionally rigid component is prevented. If, for example, two tensionally rigid components opposite a pivot axis are fixed in a stretched or tensioned position, the lower part cannot be allowed to pivot relative to the upper part about this pivot axis. If only one tensionally rigid component is fixed, pivoting is possible in the direction that causes a shortening or compression of the tensionally rigid component. The pivoting can also be blocked by maximum prestressing of the elastic component or the elastic region or the elastic restoring element, at which elastic deformation of the restoring element is no longer possible without destroying the restoring element.

The restoring element itself does not have to be exclusively elastic; it can also be coupled with an elastic component or an elastic element. By means of an elastic restoring element, it is possible to set the prestressing and, in addition, to fix the upper part on the lower part. The higher the prestressing force, the greater the restoring force with the same deflection about the corresponding pivot axis.

In one embodiment, the restoring element is detachably fastened to the upper part and/or the lower part, as a result of which it is easy, for example, to remove the upper part from the lower part or to set different restoring forces or prestresses or to replace a restoring element in the event of wear.

In one embodiment, the restoring element or the restoring elements is/are assigned an adjustment device for setting the restoring force. The restoring force can be set, for example, by the adjustment device being mounted in a movable and fixable manner on the restoring element, the upper part and/or the lower part. The effective length, for example, of an elastic belt or of an elastic band can be shortened or extended by the adjustment device. For example, through the displacement of a fastening point on the upper part and/or the lower part, an elastic component or a spring can be stretched or relaxed in order to obtain different restoring forces. If several adjustment elements are present, each can be set and adjusted individually in order to permit adaptation to different situations and/or different users of the orthopedic joint device.

The adjustment device can be mounted so as to be movable in discrete steps or continuously on the upper part and/or the lower part. Continuous displacement permits finer and more individual setting than adjustment in discrete steps, but it may require greater design effort or increased motor skills on the part of the user. An elastic band or a belt with an elastic component can have a form-fit element, for example a hook, which engages in a correspondingly shaped form-fit element, for example a projection or a hole in the upper part or lower part. The restoring element or a component connected thereto can be, for example, a part of a hook-and-loop closure, which can be individually adjustable and fixable at a corresponding location of the upper part and/or the lower part. The adjustment device can also be designed, for example, as a slide, which is mounted on the restoring element, the upper part and/or the lower part.

The adjustment device can be motor-driven, which in particular facilitates automated adjustment and adaptation of the restoring force if the motor drive is coupled to a sensor device or a control device which activates and deactivates the drive on the basis of sensor data.

In one embodiment, at least one limiting device for limiting the pivoting movement about at least one of the pivot axes is mounted adjustably on the restoring element, upper part and/or lower part. The limiting device can be formed as a slide or form-fit element which is fastened and fixable, for example, to the flexible, tensionally rigid element, which is formed in particular as a belt, band, cable or the like or portion of the restoring element. The tensionally rigid element can be part of or fastened to the restoring element. By way of the limiting device, it is possible to set the maximum pivoting angle about the respective pivot axis. This can be done, for example, by combining elastic materials with non-elastic materials. The restoring element can be formed, for example, as an elastic belt which is arranged on a flexible, non-elastic or tensionally rigid belt, wherein the non-elastic belt forms a loop. If the restoring element is extended, the non-elastic belt tightens and, in the case of complete stretching, limits further pivoting about the corresponding pivot axis. In principle, the use of exclusively elastic restoring elements is also provided and possible, in which case the maximum limit can be achieved by reaching the maximum degree of stretching, for example of an elastic band or elastic belt. The limiting device can form or cover a section of the restoring element. In the embodiment as a slide or form-fit element, the limiting device can be arranged, for example, to be movable along the longitudinal extent of the restoring element or of the tensionally rigid element and can be arranged to be fixable in the desired position. Alternatively or in addition, the limiting device is arranged on the upper part or the lower part and causes a coupling or positive locking of upper part or lower part to the restoring element or the tensionally rigid component thereof or thereon.

The restoring element in conjunction with the optionally provided limiting device fastens the upper part to the lower part and prevents a translational displacement of upper part and lower part away from each other beyond an intended dimension. The restoring element or the restoring elements provide a restoring force against a pivoting movement about each pivot axis so that, independently of the pivoting about any desired pivot axis, the starting position is adopted again when external forces are not applied.

Exemplary embodiments of the invention are explained in more detail below on the basis of the figures. In the figures:

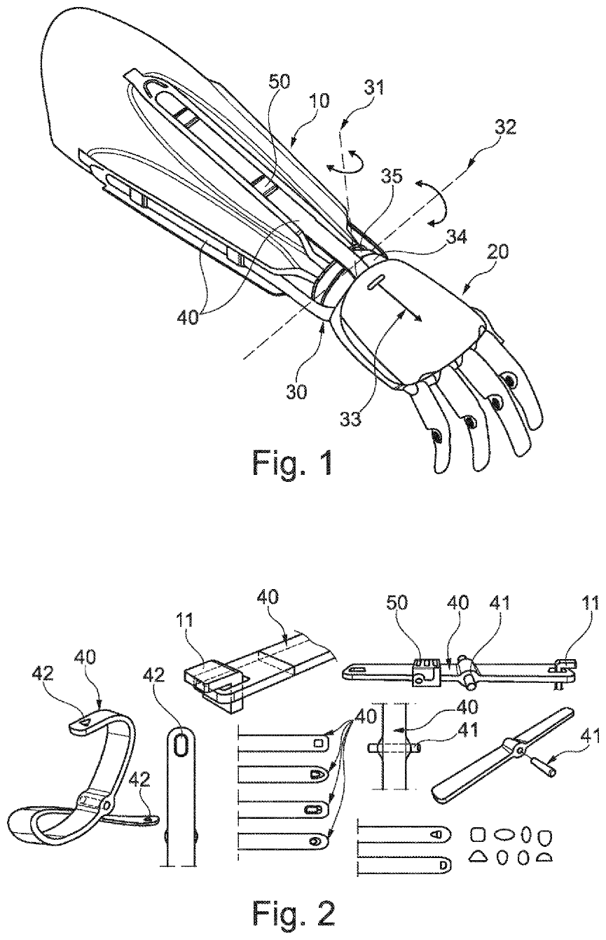

FIG. 1 shows a perspective view of a joint device;

FIG. 2 shows detailed views of a restoring element and of an adjustment device;

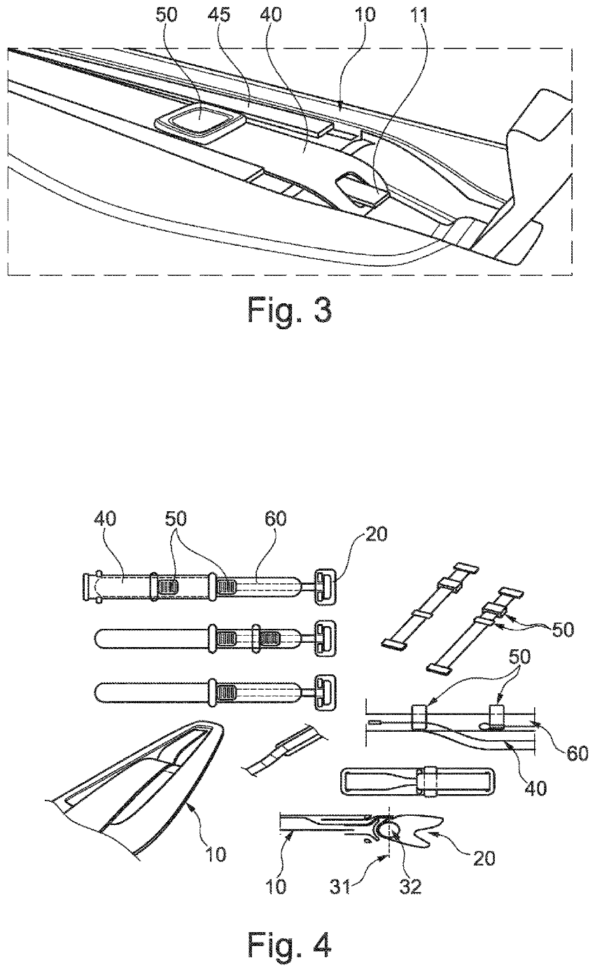

FIG. 3 shows a detailed view of an assembled restoring element;

FIG. 4 shows detailed views of a restoring element according to a further embodiment;

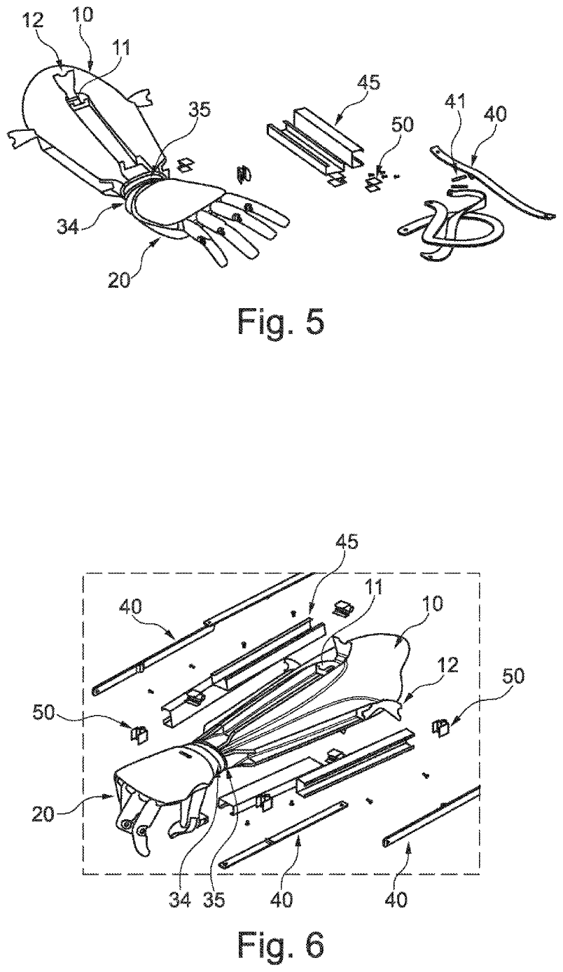

FIG. 5 shows a view of a partially disassembled prosthetic arm;

FIG. 6 shows an exploded view of a prosthetic arm;

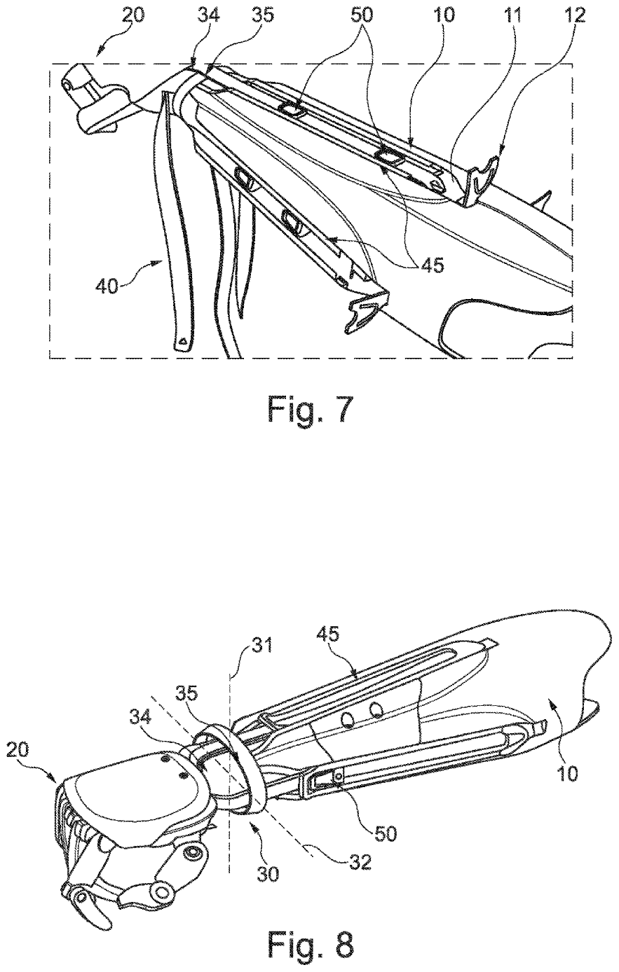

FIG. 7 shows a detailed view of a partially assembled prosthetic arm;

FIG. 8 shows a perspective view of a variant;

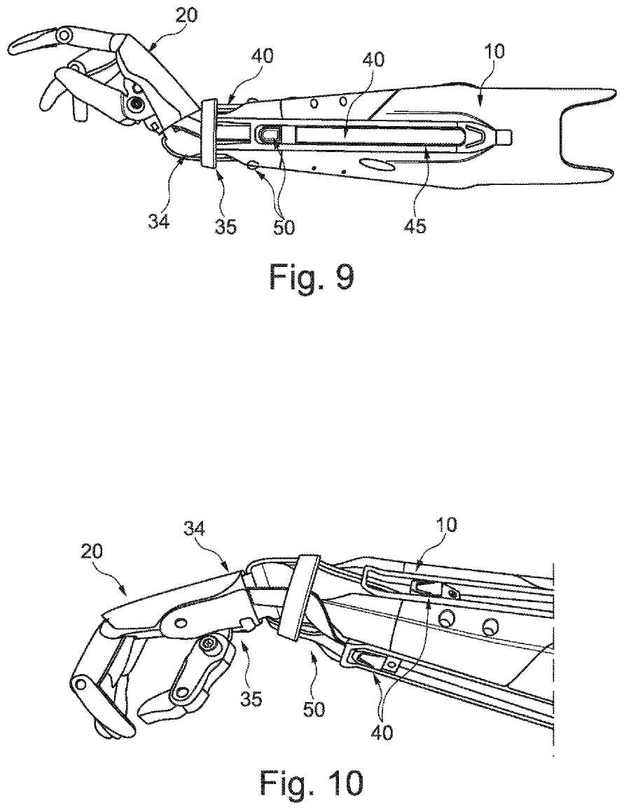

FIG. 9 shows a side view according to FIG. 8, with an extended prosthetic hand;

FIG. 10 shows a view of the embodiment according to FIG. 8 with a prosthetic hand turned about several axes;

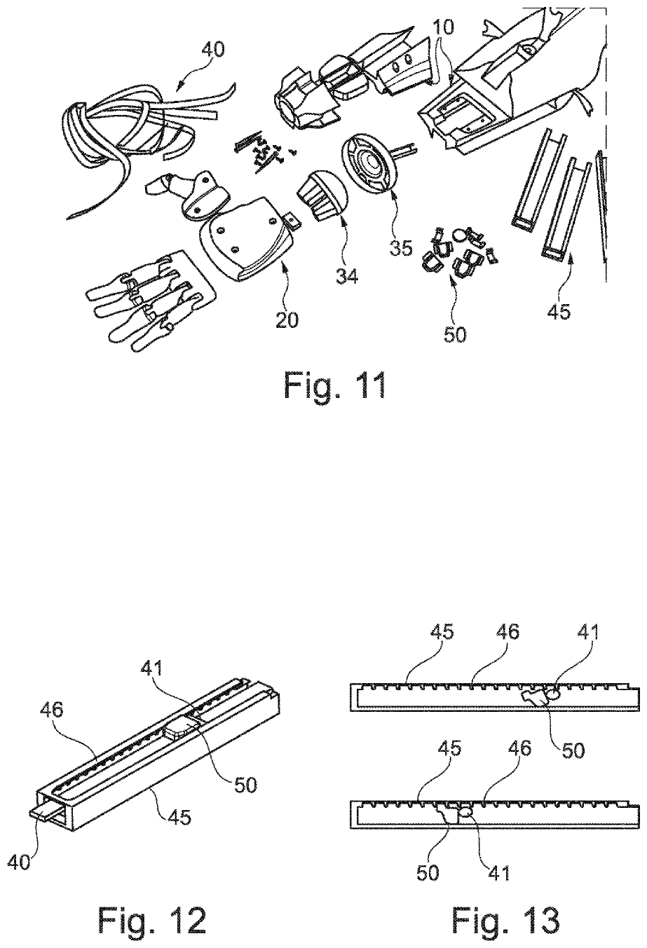

FIG. 11 shows a view of individual parts of the embodiment according to FIG. 8;

FIG. 12 shows a detailed view of a guide rail;

FIG. 13 shows two cross-sectional views of the guide rail;

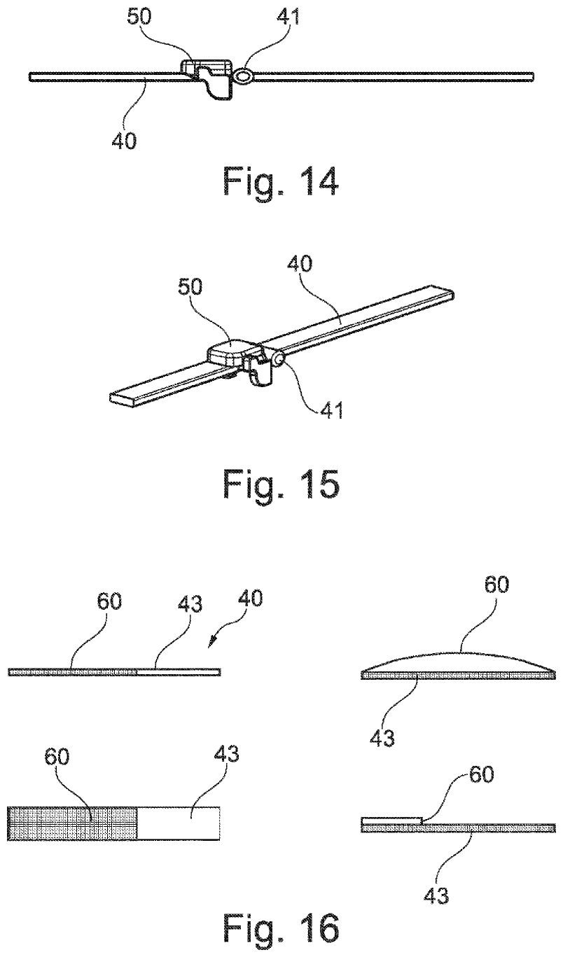

FIGS. 14 and 15 show detailed views of the restoring element and of the adjustment device;

FIG. 16 shows individual views of the restoring element; and

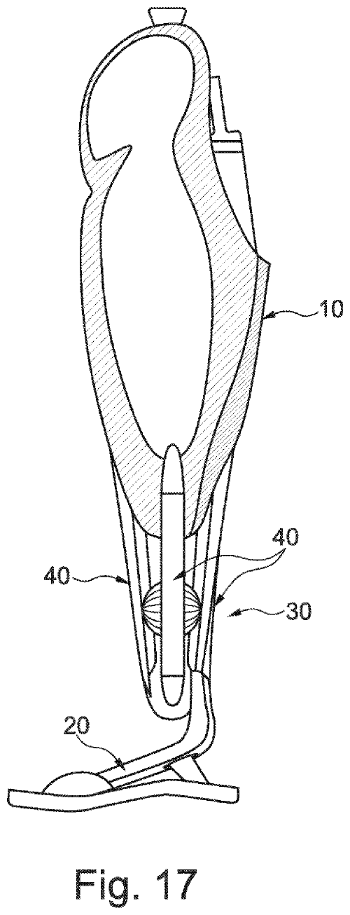

FIG. 17 shows a variant of a prosthesis of the lower limb.

FIG. 1 shows a perspective view of a prosthetic arm having an upper part 10, in the form of a forearm shaft, and a lower part 20 in the form of a prosthetic hand. Between the upper part 10 and the lower part 20, a joint 30 is formed, so that the upper part 10 and the lower part 20 together form an orthopedic joint device. The upper part 10 is of a multi-part design and, together with the lower part 20, forms at least two pivot axes 31, 32, about which the lower part 20 can be pivoted relative to the upper part 10. In the exemplary embodiment shown, the first pivot axis 31 allows a pivoting of the lower part 20 or of the prosthetic hand about an axis substantially perpendicular to the palm base, which corresponds to a pivoting in a radial direction or ulnar direction within the plane of the palm. A second joint axis 32 extends substantially perpendicular to the first pivot axis 31 and allows a flexion and extension of the lower part 20 relative to the upper part 10, so that the prosthetic hand can be pivoted in a palmar direction and dorsal direction from the illustrated starting position or neutral position.

In the position shown, the lower part 20 is in the starting position and is held in this starting position by a total of four restoring elements 40. In the embodiment shown, the restoring elements 40 are formed as elastic belts or bands which are fastened to the upper part 10 with their respective proximal end and to the lower part 20 with their respective distal end. The restoring elements 40 are guided in the upper part 10 in guide rails, which will be explained in more detail later. The restoring elements 40 are allocated several adjustment devices 50, by which it is possible to adjust the effective length of the restoring elements 40 and/or the restoring force or prestressing force of the restoring elements 40.

In the embodiment shown, the joint 30 is in the form of an ellipsoid joint or condyloid joint and has a joint head 34, which is formed on the proximal end of the lower part 20. A correspondingly formed joint socket 35 is formed or arranged at the distal end of the upper part 10 and permits a pivoting movement about the two pivot axes 31, 32. In addition, the set-up with the joint socket 35 and the joint head 34 prevents a translational displacement of the lower part 20 relative to the upper part 10 in the proximal direction, that is to say in the direction of the upper part 10, and also in directions perpendicular thereto, that is to say in the radial/ulnar direction and in the dorsal/palmar direction. Only one translational degree of freedom 33 is enabled by the joint 30, namely the displacement in the distal direction, so that the prosthetic hand or the lower part 20 can be moved away from the upper part 10 or removed. Since this is not desired during the normal operation of the prosthetic hand, the restoring elements 40 are prestressed such that the restoring elements 40 hold the prosthetic hand or the lower part 20 prestressed within the joint 30 and counteract a displacement in the distal direction. The restoring elements 40 thus fasten the prosthetic hand 20 to the prosthesis shaft displaceably within the joint 30.

The two pivot axes 31, 32 of the joint 30 are oriented perpendicular to each other in the illustrated exemplary embodiment. In the design of the joint 30 as an ellipsoid joint, it is possible that, with a large radius of the joint surfaces, a rotatability about the longitudinal extent of the upper part 10 is possible, so that the lower part 20 can perform several rotational movements or a combined rotation movement. In this case, the lower part 20 is moved in the direction of the translational degree of freedom 33 in the distal direction and thus at least partially removed from the joint socket 35. On account of the restoring force of the restoring elements 40, the joint head 34 remains pressed at the corresponding support points or support lines on the joint socket 35.

In the exemplary embodiment according to FIG. 1, a total of four restoring elements 40 are arranged lying opposite each other in pairs. All of the restoring elements 40 are prestressed, so that the illustrated starting position is maintained when no external forces or moments are applied. The prestressing of the respective restoring element 40 is adjustable, so that it is possible, for example, to provide a flexion movement with a higher resistance than an extension movement.

As an alternative to the mutually opposite, elastic restoring elements 40, which act solely on tensile force and are designed as flexible and elastic straps or bands, pull-push elements can also be arranged on and fastened to the upper part 10 and the lower part 20, which elements apply a corresponding restoring force to a pivoting movement about the two pivot axes 31, 32, proceeding from a starting position. The restoring force can then be exerted as a compressive force or tensile force, depending on the direction in which a displacement takes place. In the case of pull-push elements as restoring elements 40, only two restoring elements are then required; there is also the possibility of a plurality of such restoring elements 40 being formed and arranged on the orthopedic joint device. It is likewise possible that only one restoring element 40 is present and fastened to the upper part 10 and the lower part 20, which element exerts a restoring force in the direction of the starting position in the case of pivoting both about the first pivot axis 31 and about the second pivot axis 32.

FIG. 2 shows detailed views of the restoring element 40 or restoring elements 40, and also their assembly position and their structure. The respective restoring element 40 is designed as a belt made of an elastomer material or with an elastomer material, which belt has a recess 42 at each of its ends, with which recesses the respective restoring element 40 can be fastened with form-fit engagement to the upper part 10 or lower part 20. FIG. 2 shows just one fastening device or fastening element 11 of the upper part 10, designed as an angle bracket or hook and formed on or fastened to the upper part 10. A corresponding form-fit element is arranged or formed on the lower part (not shown) and is brought into engagement with the opposite recess 42. The recesses 42 can have different shapes, which are shown in FIG. 2, for example square, oval, round, rectangular, triangular, oval, polygonal or rounded recesses.

Between the two recesses 42 located at the ends, a thickened region is formed with a through-opening for a securing pin 41. The through-opening may have been produced in a primary forming process or may be formed by fastening a second layer of material. The securing pin 41 extends transversely to the longitudinal extent of the restoring element 40 and projects laterally from it. By means of the securing pin 41, the restoring element 40 is prevented from contracting beyond a specified point. The securing pin 41 can provide a safeguard in the event of complete unlocking of the adjustment device 50 and can guarantee a minimum prestressing of the restoring elements 40. In one embodiment, the securing pin 41 represents the abutment for the adjustment device 50. In the exemplary embodiment shown, the adjustment device 50 is formed as a slide, which is arranged on the restoring element 40 in a displaceable and fastenable manner. The adjustment device 50 can be moved either continuously or in discrete steps along the longitudinal extent of the restoring element 40 and fixed, in order to adjust the respective restoring force of the respective restoring element 40. The adjustment device 50 can be supported, either via the securing pin 41 or via other form-fit elements or force-fit elements, in a guide rail or on the upper part 10 and can change the effective length of the restoring elements 40.

FIG. 3 shows a detailed view of the restoring element 40 in an installed state in the region of the fastening to the upper part 10. The fastening element 11 is fixed in a holder on the upper part 10 and can be covered and closed by a flap. The restoring element 40 is hooked with the recess 42 into the fastening element 11 and guided along in a guide rail 45. Within the guide rail 45, the adjustment device 50 is arranged in the form of the slide which, on the one hand, clamps the restoring element 40 or is otherwise fixed thereon and, on the other hand, is fixed in the rail 45, for example clamped or hooked. The proximal part of the restoring element 40 is thus inactivated, so that the effective length of the restoring element 40 is shortened. Thus, during the displacement of the upper part 10 relative to the lower part 20, a greater relative deformation of the restoring element 40 is effected in the distal portion, so that a greater restoring force is provided for the same deflection.

FIG. 4 shows a variant of the guiding and fastening of the restoring element. In addition, the orthopedic joint device has a limiting device 60 in the form of a flexible and non-elastic belt or band, which is likewise coupled to an adjustment device 50. The restoring element 40 and the limiting device 60 are guided substantially parallel and are fastened on the one hand to the upper part 10 and on the other hand to the lower part 20. By means of a corresponding setting of the adjustment devices 50, it is possible to set both the restoring force or prestressing and the maximum attainable pivot angle. Both variables can be set independently of each other and can accordingly be used both for flexion and extension and for rotation about the pivot axis 31. The two pivot axes 31, 32 are indicated in the view at the bottom of FIG. 4; the second pivot axis 32 runs perpendicular to the plane of the drawing, the first pivot axis 31 perpendicular thereto within the plane of the drawing.

It is likewise possible to arrange two restoring elements 40 with two adjustment devices 50 for example on the top of the upper part 10, in order to ensure easy accessibility. One of the restoring elements 40 serves to provide a restoring force upon flexion of the lower part 20, the other upon extension. Corresponding limiting devices 60 can accordingly also be arranged on the easily accessible top and coupled to adjustment devices 50. The fastening to the upper part 10 then takes place at fastening points on mutually opposite sides of the pivot axis 32 or 31.

In FIG. 5, the embodiment according to FIG. 1 is shown in a partially disassembled state. The restoring elements 40 in the form of elastic bands with optionally different lengths or elasticities are each provided with recesses at the ends in order to be able to be fixed to the fastening elements on the upper part 10 and the lower part 20. The fastening is reversible, so that the restoring elements 40 can be easily replaced and adapted. The securing pin 41 can be inserted into a corresponding receptacle in the restoring element 40. The guide rails 45 can also be seen, with the associated adjustment devices 50 which are mounted movably within the guide rails 45, in order to be locked with form-fit or force-fit engagement at different points along the longitudinal extent of the guide rails 45. The adjustment device 50 can either be fixed on the restoring element or can be clamped in a movable manner thereon. The upper part 10 has receptacles for the total of four guide rails 45, at the proximal end points of which the fastening devices 11 and the cover flaps 12 are arranged or inserted. At the distal end of the upper part 10, the joint socket 35 of the ellipsoid joint 30 is formed, while, at the proximal end of the lower part 20, the joint head 34 is formed in a barrel-like or egg-shaped configuration. Both the joint socket 35 and the joint head 34 can be arranged exchangeably on the respective components. As an alternative to the ellipsoid joint shown or to the barrel shape, the components of the joint 30 can also be formed as a ball joint with a flat joint socket 35.

In FIG. 6, the individual components of the embodiment according to FIG. 1 are shown in an exploded view. The guide rails 45 with the adjustment devices 50 are inserted into the recesses both at the top and bottom and also medially and laterally on the upper part 10 and fixed therein. The restoring elements 40 in the form of elastic bands or belts are fastened in a form-fitting and reversible manner on the upper part 10 and the lower part 20 and guided in the guide rails 45. Before the restoring elements 40 are fastened, the adjustment devices 50 are pushed onto the restoring elements 40 and then inserted into the guide rails 45. Two adjustment devices 50 are provided per restoring element 40 and are positioned on both sides of the housing of the securing pin 41 in order to be able to adjust different restoring forces continuously or in steps. The joint socket 35 is arranged or formed on a support part and is inserted into the shaft of the upper part 10.

FIG. 7 shows the partially assembled state of the orthopedic joint device in the form of the prosthetic arm. A restoring element 40 on the top of the upper part 10 is already form-fittingly locked in the fastening device 11 and guided with the adjustment devices 50 within the guide rail 45. The flap 12 is not yet closed. The other three restoring elements 40 are not yet inserted and fixed in the guide rails 45 and the adjustment devices 50. It can be seen that all the restoring elements 40 are fastened to the lower part 20, such that two restoring elements 40 lie opposite each other in pairs. This ensures that, from a starting position, a pivoting movement can take place in both possible directions of pivoting about the respective rotational degree of freedom and a restoring movement can take place with a corresponding restoring force.

FIG. 8 shows a further embodiment of the orthopedic joint device, likewise in the form of a prosthetic arm. The basic set-up does not differ from the embodiment according to FIG. 1. Here, too, there are two pivot axes 31, 32, which are oriented substantially perpendicular to each other in the ellipsoid joint 30. The lower part 20 can thus execute two pivoting movements about the pivot axes 31, 32, optionally also a combined movement. The lower part 20 is movably fastened to the upper part 10 via a total of four restoring elements 40. The restoring elements 40 are designed as elastic bands or belts and, in the exemplary embodiment shown, are guided in a double layer within the guide rails 45. The adjustment devices 50 are likewise guided in the guide rails 45 and limit the effective length of the respective restoring elements 40 between the adjustment device 50 and the lower part 20. The restoring elements 40 are guided in the region of the joint socket 35 within a circumferential ring in order to protect the restoring elements 40 during a movement of the joint head 34 within the joint socket 35 and in order to prevent the restoring elements 40 from twisting about themselves or trapping other objects.

FIG. 9 is a side view showing the prosthetic arm with upper part 10 and lower part 20 in an extended position. In this case, a pivoting movement takes place about the second pivot axis, axis 32, which is shown in FIG. 8, such that the back of the prosthetic hand is moved in the direction of the upper part 10. This causes the restoring element 40 fastened to the underside to be stretched, while the restoring element 40 on the top is relaxed. The medially and laterally arranged restoring elements 40 are likewise moved slightly; the deformation contributes only slightly, if at all, to an elongation and thus to the generation of a restoring force in the direction of the starting position according to FIG. 9. Through the ring, which is formed around the outside of the joint socket 35, the restoring elements 40 remain aligned with the guide rails 45, so that safe and reliable operation can take place.

FIG. 10 is an oblique plan view showing a combined movement of the lower part 20 relative to the upper part 10. The joint head 34 on the lower part 20 is rotated both about the second pivot axis 32 and about the longitudinal extent of the upper part 10, such that the joint head 34 has lifted slightly out of the joint socket 35. This can be seen from the twisted restoring elements 40. In the absence of external forces, the lower part 20 is moved back to the starting position relative to the upper part 10, so that the joint head 34 is again fully received within the joint socket 35.

FIG. 11 shows a view of individual parts of the embodiment according to FIGS. 8 to 10. The prosthetic hand as the lower part 20 is constructed in several parts and has a base body similar to a palm, with attached fingers and a thumb module.

Arranged at the proximal end of the lower part 20 is the joint head 34, which is formed separately and which can either be fixed to the base body permanently by integral bonding or exchangeably by form-fit engagement. The upper part 10 is likewise constructed in several parts and has receptacles for the rails 45 and for the joint socket 35. The components are pluggable and fixable and can be fastened to each other, for example, by screws or similar fastening devices. Likewise, the guide rails 45 can be fixed in the upper part 10 via screws or other fastening elements. Both the joint head 34 and the joint socket 35 are formed as a condyloid joint or ellipsoid joint, so that an activity can be easily achieved in several planes about several axes, in the present case about two axes. The main mobility is about the second pivot axis 32 along the longitudinal extent of the ellipsoid joint.

FIG. 12 shows a perspective individual view of the guide rails 45 with the restoring element 40 and the adjustment device 50. In this embodiment, the guide rail 45, at its upper, inwardly projecting rail parts, has downwardly directed form-fit elements 46 in the shape of teeth, which can be seen more clearly in the views in FIG. 13. The views in FIG. 13 show a cross-sectional representation without the restoring element. The adjustment device 50 is fixed to the restoring element 40 and is pressed upward against the form-fit elements 46 by the tensile stress in the prestressed restoring element 40. The adjustment device 50 has projections, like the securing pin, protruding laterally over the restoring element 40 and can be disengaged from the form-fit elements 46 by pushing down. The adjustment device 50 is then moved to the desired position and released so that the lateral projections, as shown in the lower view in FIG. 13, come into engagement again with the form-fit elements 46.

FIGS. 14 and 15 show a restoring element 40 with an adjustment device 50 and with a securing pin 41 arranged movably on the restoring element 40. The securing pin 41 is steplessly movable and is designed to be able to be fixed on the restoring element 40. The securing pin 41 limits the effective length of the restoring element 40 or allows a change in the prestressing of the elastic element of the restoring element 40. The adjustment device 50 has an operating face which protrudes over the guide rail 45 (not shown) and which can be guided movably within the central recess of the guide rail 45. To the side thereof, the projections shaped correspondingly to the form-fit elements 46 protrude beyond the width of the restoring element 40, so as to be able to come into engagement with the form-fit elements 46.

FIG. 16 shows individual views of the restoring element 40, in which both an elastic element 43 and a limiting device 60 are shown. The two figures on the left show, in a side view and in a plan view, a restoring element 40 with a limiting device 60 made of a flexible, non-elastic material, which is adjoined by an elastic element 43. The elastic element 43 likewise has the form of a belt and is arranged, for example, proximally to the limiting device 60. If an adjustment device (not shown) is moved into the region of the limiting device 60 and locked there, for example by form-fit engagement, with one of the guide rails, no elastic displacement of the lower part relative to the upper part takes place in the pulling direction. However, displacement in the direction of the elastic element 43 or the elastic section is possible. If the adjustment device (not shown) is moved into the region of the elastic element 43 or the elastic section 43, a pivoting of the upper part relative to the lower part can take place on account of the elongation of the elastic element 43.

Two further embodiments of the restoring element 40 are shown in the two side views on the right of FIG. 16, which show a parallel arrangement or parallel connection of an elastic element 43 and a limiting device 60. In the upper view, the two components are connected to each other at their two ends, the limiting device 60 being longer than the elastic element 43. An expansion of the elastic element 43 is then only possible until the belt as limiting device 60 is stretched to the maximum. In the lower view, the limiting device 60 is shorter than the elastic element 43 and, for example, is sewn onto the top of the elastic element 43, thereby achieving the same effect as in the view on the left.

FIG. 17 shows a perspective view of a prosthesis device for a lower limb. The prosthesis forms an orthopedic joint device having an upper part 10 in the form of a prosthetic lower leg with a proximal fastening device for securing the prosthetic lower leg to a thigh socket. Arranged within the prosthetic lower leg is a prosthetic knee joint with an actuator or a damper. In the distal region of the upper part 10, a joint 30 is formed which connects a prosthetic foot as lower part 20 pivotably to the upper part 10. The joint 30 is formed, for example, as a ball joint or ellipsoid joint, so that two pivot axes (not shown) are formed. The prosthetic foot as lower part 20 can execute an anterior and posterior pivoting movement within the sagittal plane and a medial and lateral pivoting movement within the frontal plane. On the outside of both the upper part 10 and the lower part 20, there are a total of four restoring elements 40, which are constructed as described above and prestress the lower part 20 elastically in the direction of the upper part 10. A pivoting movement from the illustrated starting position in one of the possible directions of pivoting has the effect that at least one restoring element 40 is stretched, as a result of which a restoring force is exerted in the direction of the starting position. The restoring force is adjustable, as described above. In principle, a limiting device for limiting the maximum pivoting in the respective direction is also possible and provided.

1. An orthopedic joint device, comprising:
an upper part;
a lower part;
a joint positioned between the upper part and the lower part which forms at least two pivot axes which are not oriented parallel to each other; and
at least one restoring element fastened to the upper part and the lower part, wherein the restoring element comprises an elastic element which, during a pivoting movement of the upper part relative to the lower part from a starting position, exerts a restoring force in a direction of the starting position.

2. The orthopedic joint device as claimed in claim 1, wherein the joint comprises a joint head and a joint socket and is formed as a ball joint or ellipsoid joint.

3. The orthopedic joint device as claimed in claim 1 wherein the at least two pivot axes are oriented perpendicular to each other.

4. The orthopedic joint device as claimed in claim 1 wherein the joint has at least one translational degree of freedom.

5. The orthopedic joint device as claimed in claim 1 wherein the at least one restoring element comprises several restoring elements, each of which are fastened to the upper part and the lower part and are arranged to be active in different directions.

6. The orthopedic joint device as claimed in claim 1 wherein the at least one restoring element is formed as a tension element, belt, band or elastomer, and wherein the at least one restoring element comprises at least one tensionally rigid, flexible component.

7. The orthopedic joint device as claimed in claim 1 wherein the at least one restoring element is fastened detachably to the upper part and/or the lower part.

8. The orthopedic joint device as claimed in claim 1 wherein the at least one restoring element is assigned an adjustment device for adjusting the restoring force.

9. The orthopedic joint device as claimed in claim 8, wherein the adjustment device is mounted in a movable and fixable manner on the at least one restoring element, the upper part and/or the lower part.

10. The orthopedic joint device as claimed in claim 8 wherein the adjustment device is mounted so as to be movable in discrete steps or continuously on the upper part and/or the lower part.

11. The orthopedic joint device as claimed in claim 8 wherein the adjustment device is driven by motor.

12. The orthopedic joint device as claimed in claim 1 further comprising at least one limiting device for limiting a pivoting movement about at least one of the at least two pivot axes is mounted adjustably on the at least one restoring element, the upper part, and/or the lower part.

13. The orthopedic joint device as claimed in claim 12, wherein the at least one limiting device is designed as a flexible, tensionally rigid element.

14. The orthopedic joint device as claimed in claim 1 wherein the orthopedic joint device is designed as a prosthetic wrist or as a prosthetic ankle joint.

15. The orthopedic joint device as claimed in claim 1 wherein the at least one restoring element fastens the upper part to the lower part.

16. The orthopedic joint device as claimed in claim 1 wherein the at least one restoring element exerts a restoring force in a direction of the starting position during a pivoting movement about each of the at least two pivot axes.

17. The orthopedic joint device of claim 5 wherein the different directions comprise opposite directions.

18. The orthopedic joint device of claim 13 wherein the flexible, tensionally rigid element is a belt, a band, a cable, a slide element, or a form-fit element.
